(19) United States
(12) Patent Application Publication
(10) Pub. No.: US 2001/0045375 A1
(43) Pub. Date: Nov. 29, 2001

(54) Apparatus and method for conversion of hydrocarbon feed streams into liquid products

(76) Inventors: Johannes H.J.S. Thijssen, Cambridge, MA (US); Carole J. Read, Watertown, MA (US); Robert S. Weber, Lincoln, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/766,699

(22) Filed: Jan. 22, 2001

Related U.S. Application Data
(60) Provisional application No. 60/177,852, filed on Jan. 24, 2000.

(51) Int. Cl. C10G 45/00; C07C 27/06; B01J 8/00

(52) U.S. Cl. 208/211; 422/187

(57) ABSTRACT

Disclosed are apparatus and methods for conversion of hydrocarbon feed streams into liquid products. One embodiment of an apparatus includes a pressure vessel that contains a synthesis gas production device, a synthesis gas conditioning device and a synthesis gas conversion device wherein the synthesis gas production device and the synthesis gas conditioning device are nested within the synthesis gas conversion device. One embodiment of a method includes providing a hydrocarbon feed stream and producing a synthesis gas stream from the hydrocarbon feed stream in a synthesis gas production device. Subsequently, the synthesis gas stream is conditioned by removing heat from the synthesis gas stream through a first hollow body into a reactant feed stream that is then fed into the synthesis gas production device. Finally, the synthesis gas stream is converted to form a liquid product stream.

### RELATED APPLICATION

This application is entitled to the benefit of earlier filed U.S. Provisional Patent Application Ser. No. 60/177,852, filed Jan. 24, 2000, under 35 U.S.C. 119(e), the entire disclosure of which is hereby incorporated by reference herein.

### FIELD OF THE INVENTION

The present invention relates to apparatus and methods for processing hydrocarbons. More specifically, the invention relates to apparatus and methods for converting a hydrocarbon feed stream into a liquid product stream.

### BACKGROUND OF THE INVENTION

Known steps of processes typically used to convert substantially gaseous hydrocarbons to liquid products include producing synthesis gas, e.g., by partial oxidation and finishing using catalytic steam reforming, conditioning or cooling of the formed synthesis gas, and subsequently converting of the synthesis gas to liquid products by, e.g., a Fischer-Tropsch reaction. The Fischer-Tropsch reaction, developed in the 1920s, typically is carried out in a three-phase catalytic reactor and produces a mixture of long-chain saturated hydrocarbons. Alternative conversion processes include the methanol-to-gasoline process and other conversion technologies focused on the production of other products such as methanol or dimethyl ether.

These processes typically are carried out in a series of separate pressure vessels. Because these processes typically occur at relatively high temperatures and pressures, e.g., synthesis gas production typically is carried out at pressures above about 25 bara and temperatures above about 800° C. and which also produces a large amount of heat, the construction of pressure vessels and the jacketed connections between them for the individual steps often are prohibitively expensive. This is particularly the case for the conversion of hydrocarbon feed streams on relatively small scales, e.g., for plants processing less than about 100 million standard cubic feet of natural gas per day. Consequently, natural gas and other substantially gaseous hydrocarbon reservoirs, e.g., in oil reserves, coal deposits, or formed during mining operations, often are flared or vented. This not only is a waste of natural resources, but also results in the emission of pollutants into the environment.

Moreover, the large number of interconnected pressure vessels required in conventional gas to liquid processes generally are impractical in certain applications, e.g., on an oceanic drilling platform or a floating, production, storage, off-loading vessel (FPSO), because space is at a premium where such facilities exist. In addition, the piping and pressure vessels must all be manufactured and constructed to high safety standards since the apparatus are susceptible to damage from the harsh environment and/or other machinery in the vicinity.

### SUMMARY OF THE INVENTION

What is needed are modular, compact, and cost effective apparatus and methods for converting hydrocarbon feed streams into liquid products. Modular, compact and cost effective apparatus and methods for converting hydrocarbon feed streams into liquid products have been discovered wherein heretofore separately operating processes are combined and integrated within a single pressure vessel. These processes include, but are not limited to, synthesis gas production, synthesis gas conditioning, and synthesis gas conversion.

As disclosed herein, the specific combination of devices which carry out these processes allows for a compact and readily mobile conversion plant. Also, exposure to damage from the environment is minimized because certain individual devices and their interconnections are combined within a single pressure vessel. Furthermore, the costs of construction are minimized because the individual devices are organized in a nested arrangement such that those processes requiring the highest temperatures and severe atmospheres are centrally located. In this design, the outside shell is rated for full system pressure and the expense of jacketing each device within its own pressure vessel is eliminated for the nested devices, which do not need to be rated for full system pressure. In addition, the quantity of exotic metallurgy needed for the high temperature and severe environment is minimized, further reducing capital cost. Similarly, the construction standards for interconnections between the devices are minimized since the interconnections do not need to be individually pressure jacketed because they are internal to the pressure vessel. Accordingly, the inclusion of certain of these processes within one pressure vessel presents a significant cost savings relative to enclosing each process separately within its own pressure vessel.

In one aspect of the present invention, an apparatus for conversion of a hydrocarbon feed stream into a liquid product stream is disclosed that includes a pressure vessel, including a synthesis gas production device, a synthesis gas conditioning device, and a synthesis gas conversion device. These devices are in fluid communication with one another, with the synthesis gas production device and the synthesis gas conditioning device nested within the synthesis gas conversion device.

In other words, the invention disclosed herein includes an apparatus for conversion of a hydrocarbon feed stream into a liquid product stream that includes a pressure vessel including a means for producing synthesis gas, a means for conditioning synthesis gas and a means for converting synthesis gas that are in fluid communication with each other such that the means for producing synthesis gas and the means for conditioning synthesis gas are nested within the means for converting synthesis gas.

Another aspect of the invention is a method for conversion of a hydrocarbon feed stream into a liquid product stream including the steps of: (a) providing a hydrocarbon feed stream; (b) producing a synthesis gas stream from the hydrocarbon feed stream in a synthesis gas production device; (c) conditioning the synthesis gas stream, wherein the conditioning step includes: (c′) removing heat from the synthesis gas stream through a first hollow body into a reactant feed stream passing through the first hollow body to provide a preheated reactant feed stream, and (c″) feeding the preheated reactant feed stream into the synthesis gas production device; and (d) converting the synthesis gas stream to a liquid product stream.

Reference to the figures herein is intended to provide a better understanding of the methods and apparatus of the invention but are not intended to limit the scope of the invention to the specifically depicted embodiments. The drawings are not necessarily to scale, emphasis instead being placed upon illustrating the principles of the invention. Like reference characters in the respective figures typically indicate corresponding parts.

It should be understood that the order of the steps of the methods of the invention is immaterial so long as the invention remains operable, e.g., a hydrocarbon feed stream must be provided prior to the partial oxidation of the hydrocarbon feed stream. Moreover, two or more steps may be conducted simultaneously unless otherwise specified.

The foregoing, and other features and advantages of the invention, as well as the invention itself, will be more fully understood from the description, drawings, and claims which follow.

### BRIEF DESCRIPTION OF THE DRAWINGS

FIG. 1 is a high-level cross-sectional schematic view of an embodiment of an apparatus for conversion of a hydrocarbon feed stream into liquid products in accordance with the present invention.

FIG. 2 is a detailed cross-sectional schematic view of an embodiment of an apparatus for conversion of a hydrocarbon feed stream into liquid products in accordance with the present invention.

FIGS. 3A and 3B are cross-sectional schematic views of FIG. 2, taken along lines A-A and B-B, respectively.

FIGS. 4, 5 and 6 are high-level cross-sectional schematic views of alternative embodiments of apparatus for conversion of a hydrocarbon feed stream into liquid products in accordance with the present invention.

FIG. 7 is a flowchart summarizing an embodiment of a method for conversion of a hydrocarbon feed stream into liquid products in accordance with the present invention.

FIG. 8 is a flowchart summarizing another embodiment of a method for conversion of a hydrocarbon feed stream into liquid products in accordance with the present invention.

### DETAILED DESCRIPTION OF THE INVENTION

Liquid products can be produced from hydrocarbon feed streams in a cost-effective way using the apparatus and methods of the present invention. The apparatus of the present invention also is compact and modular so that it may be used as needed in applications where space is limited. In addition, the apparatus may readily be moved to various sites as needed.

By integrating synthesis gas production, synthesis gas conditioning, and synthesis gas conversion within one pressure vessel, and thus eliminating the need for separate pressure vessels for these process steps, the above advantages are realized. For example, equipment and installation costs resulting from the construction of multiple vessels, their interconnections, and safety devices are minimized. In addition, only one set of safety devices is required and external connections to other processes, if any, are significantly reduced. Moreover, the walls containing each process step within the pressure vessel do not have to be rated for the pressure differential between the pressure experienced in the process step and atmospheric pressure. Rather, the construction and materials of the walls only need account for the slight pressure variations within the pressure vessel. Furthermore, the combination of these processes and their interconnections within the pressure vessel minimizes the risk for damage to the apparatus from exposure to the environment.

Apparatus and methods of the invention are widely applicable and especially suitable for oil exploration and production applications where associated hydrocarbon gas and/or liquids are produced. In such applications, the supply of hydrocarbon feed streams may fluctuate. Accordingly, the demand for flexible, compact processes and low manufacturing costs is high. For example, in offshore applications the apparatus and methods of the present invention may be employed to convert natural gas fractions and/or natural gas liquid fractions into useful products thereby avoiding flaring of this resource. The apparatus and methods of the invention are particularly useful when the flow rate of such feed streams is less than about 100 million standard cubic feet per day. The apparatus and methods of the present invention not only provide a cost-effective alternative to flaring or release, but also enable the conservation of natural resources and minimize the release of pollutants into the atmosphere.

It is envisioned that the apparatus of the present invention may be particularly useful in remote, small-scale applications, such as on ships, scouting platforms, oil and gas production platforms, and remote land operations. Therefore, the apparatus may be scaled for ranges of capacities and sizes so that it easily may be moved or combined as necessary.

As used herein, “hydrocarbon feed stream” is understood to mean a fluid hydrocarbon feed stream that is substantially composed of hydrocarbons. Preferably, a hydrocarbon feed stream is a substantially gaseous hydrocarbon stream, e.g., natural gas, but also may be a liquid hydrocarbon stream or a combination of both gaseous and liquid hydrocarbon products. For example, the hydrocarbon feed steam may include natural gas liquids. The hydrocarbon feed stream may also include a recycle stream from one or more devices of the apparatus, e.g., gaseous by-products, that may be recycled and added to the hydrocarbon feed stream. Gaseous by-products may include, e.g., unreacted synthesis gas, and recycle may be accomplished inside or outside of the pressure vessel. The composition of a hydrocarbon feed stream typically fluctuates over time depending on the source of the stream. That is, the fractions of the specific hydrocarbons may vary as well as any non-hydrocarbon constituents such as sulfur compounds, nitrogen, water, carbon monoxide, hydrogen, and carbon dioxide.

As used herein, “liquid products” is understood to mean not only liquid hydrocarbons, but also any other liquid product that can be produced from synthesis gas such as, e.g., methanol, ethanol, dimethyl ether, light ethers, ammonia and/or alpha-olefins. Liquid products may contain unconverted reactants, gaseous byproducts or contaminants such as water, carbon dioxide, nitrogen, light hydrocarbons, light alcohols, light ethers, and light organic acids entrained therein. In addition, liquid products may contain a fraction of suspended, dissolved or otherwise dispersed solid hydrocarbon products, such as wax.

As used herein, “reactant feed stream” means a feed stream that contains reactants. Thus, a reactant feed stream may include a hydrocarbon feed stream, an oxidation gas feed stream, e.g., an air or oxygen stream, and/or an aqueous stream, e.g., a steam stream.

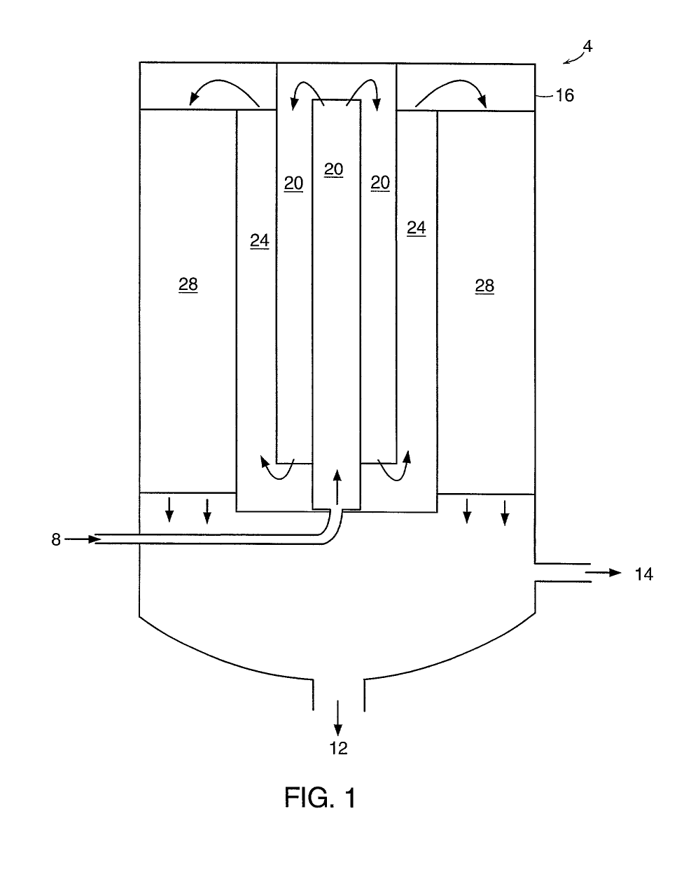

FIG. 1 is a high-level cross-sectional schematic view of an embodiment of an apparatus 4 of the present invention for conversion of a hydrocarbon feed stream 8 into a liquid product stream 12. The apparatus includes a pressure vessel 16 that generally includes a synthesis gas production device 20, a synthesis gas conditioning device 24 in fluid communication with the synthesis gas production device 20, and a synthesis gas conversion device 28 in fluid communication with the synthesis gas conditioning device 24.

As can be seen in FIG. 1, the synthesis gas production device 20 is nested within the synthesis gas conditioning device 24, and the synthesis gas production device 20 and the synthesis gas conditioning device 24 are nested within the synthesis gas conversion device 28. Thus, the synthesis gas production device 20, which typically operates at a temperature between about 700° C. and about 1400° C., preferably between about 800° C. and about 1100° C., is nested within the synthesis gas conversion device 28. The synthesis gas conversion device 28 typically operates at temperatures between about 150° C. and 400° C., preferably between about 220° C. and about 270° C. The nesting of one or more high temperature devices within one or more relatively low temperature devices in the apparatus 4 shields the exterior wall of the pressure vessel 16 from the high temperatures experienced in the high temperature devices, e.g., the synthesis gas production device 20. In FIG. 1, the nesting of the synthesis gas production device 20 inside the synthesis gas conditioning device 24 and the gas conversion device 28, shields the walls of the pressure vessel 16 from the synthesis gas production temperatures. Instead, the inner surface of the walls of the pressure vessel 16 experience the synthesis gas conversion temperatures.

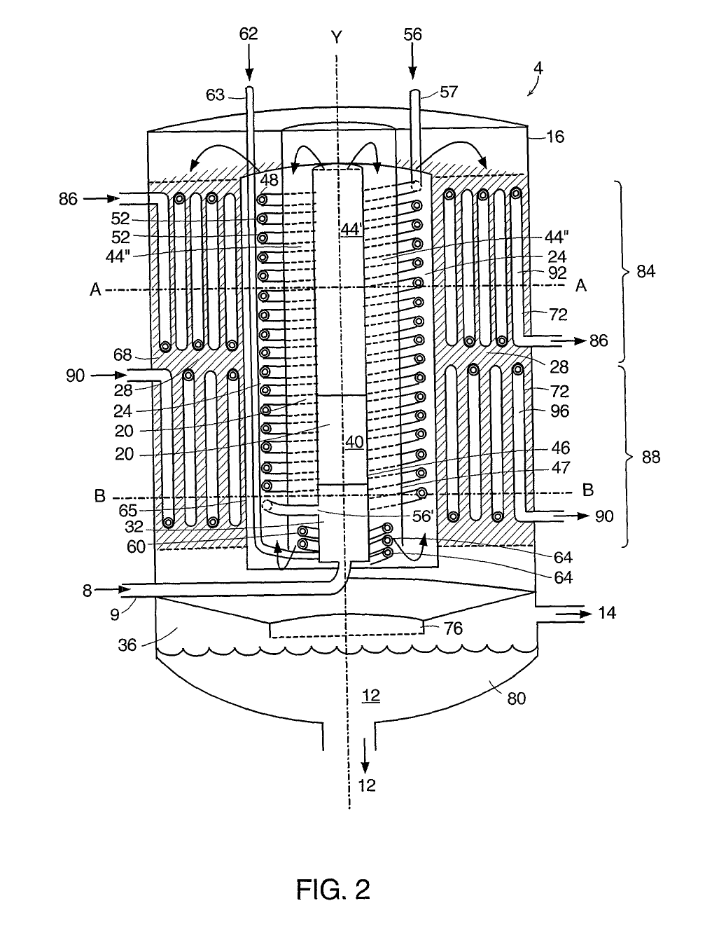

FIG. 2 is a detailed cross-sectional schematic view of an embodiment of an apparatus 4 of the present invention for the conversion of a hydrocarbon feed stream 8 into a liquid product stream 12. As in FIG. 1, the apparatus 4 includes a pressure vessel 16 that generally includes a synthesis gas production device 20, a synthesis gas conditioning device 24 in fluid communication with the synthesis gas production device 20, and a synthesis gas conversion device 28 in fluid communication with the synthesis gas conditioning device 24. The synthesis gas production device 20 and the synthesis gas conditioning device 24 are nested within the synthesis gas conversion device 28 so the same benefits described above for FIG. 1 are realized in this embodiment.

A hydrocarbon feed stream 8 is introduced into the pressure vessel through conduit 9, which is in fluid communication with a reactant gas preparation device 32. Also in fluid communication with the reactant gas preparation device 32 is an oxidation gas feed stream 56 that enters the pressure vessel 16 through conduit 57. The oxidation gas feed stream 56 enters the reactant gas preparation device 32 at inlet 56′. Preferably, the oxidation gas feed stream 56 is substantially air. Alternatively, the oxidation gas feed stream 56 may be oxygen-enriched air. An aqueous stream 62 that enters pressure vessel 16 through conduit 63 also may be introduced to the reactant gas preparation device 32 (connection not shown).

The hydrocarbon feed stream 8, the oxidation gas feed stream 56, and/or the aqueous stream 62 may be introduced to pressure vessel 16 at elevated pressure using conventional pressurization devices, such as compressors and pumps. Depending on the application, the pressure may vary from about 20 bara to about 100 bara. For example, in an application based on Fischer-Tropsch conversion, the hydrocarbon feed stream 8 and the oxidation gas feed stream 56 preferably are compressed to between about 25 bara and about 40 bara.

The reactant gas preparation device 32 is in fluid communication with a synthesis gas production device 20 through, e.g., one or more injectors (not shown). The reactant gas preparation device 32 may be used to prepare the reactant gas, i.e., the hydrocarbon feed stream 8, the oxidation gas feed stream 56, and the aqueous stream 62 for synthesis gas production. For example, the reactant gas preparation device 32 may contain devices to remove contaminants, may be used to preheat reactant gases, and/or may contain a device to pre-mix the reactant gases to desired ratios for subsequent synthesis gas production and conversion. Alternatively, a one or more of these devices may be located outside the pressure vessel 16. Preferably, the reactant gas preparation device 32 includes a preheating device and a premixing device. A contaminant removal device, such as a sulfur removal device, may be located either within the pressure vessel in the reactant preparation device 32 or outside the pressure vessel 16, to remove sulfur-containing compounds from the hydrocarbon feed stream 8. Sulfur removal devices are known and typically include a conventional sulfur sorbent, such as zinc oxide based sorbent.

The reactant gas preparation device 32 also may contain a preheating device (not shown). Preferably, the preheating device uses the enthalpy of the synthesis gas to preheat the reactant gas and cool the synthesis gas. This preheating device may include an indirect heat exchanger, e.g., a coil, tube, or finned-tube heat exchanger, to transfer heat from the aqueous stream 62 after it has passed through the second synthesis gas heat exchanger 60 (discussed below). Alternatively or additionally, the aqueous stream 62 may be introduced into the reactant gas preparation device 32 as a reactant. The reactant gas preparation device heat exchanger (not shown) is preferably a coil-type heat exchanger in fluid communication with the second synthesis gas heat exchanger 60. The reactant gas preparation device may be used to preheat the oxidant gas feed stream 56, the oxidant gas feed stream 56 mixed with the hydrocarbon feed stream 8, or the hydrocarbon feed stream 8 mixed with both the oxidation gas feed stream and the aqueous stream 62. Preferably, the reactant gas stream, regardless of its composition, is preheated to between about 250° C. and about 600° C.

Preferably, the oxidation gas feed stream 56 and the hydrocarbon feed stream 8 are introduced to the apparatus 4 at system pressure, typically from about 20 bara to about 100 bara. The hydrocarbon feed stream 8, the oxidation gas feed stream 56, and/or the aqueous stream 62 may enter the partial oxidation device separately or in a pre-mixed state. Preferably, however, the reactant gas preparation device 32 further includes a device to pre-mix the hydrocarbon feed stream 8, the oxidation gas feed stream 56, and/or the aqueous stream 62, preferably steam, at the desired ratios for subsequent synthesis gas production. The ratios are chosen so that the production ratio of hydrogen and carbon monoxide in the synthesis gas production device 20 is optimized and the production of carbon dioxide and carbon is minimized. For Fischer-Tropsch-based conversion processes based on cobalt-containing catalysts, the ratio of hydrogen to carbon monoxide preferably is about 2:1. Typically, a fuel equivalence ratio between about 3 to 4 is used, preferably between about 3.25 and 3.75. “Fuel equivalence ratio,” as used herein, means the ratio of the amount of molecular oxygen required to fully oxidize the hydrocarbon feed stream 8 to the actual amount of molecular oxygen provided.

In addition, some or all of the aqueous stream 62, preferably steam, may be added to the hydrocarbon feed stream 8 and the oxidation gas feed stream 56 in the reactant gas preparation device 32. Preferably, the aqueous stream 62 is mixed with the hydrocarbon feed stream 8 prior to the addition of the oxidation gas feed stream 56, at a ratio of molecular steam to atomic carbon of between about 0 and about 0.8, more preferably at a ratio between about 0.25 and about 0.5.

Again referring to FIG. 2, the synthesis gas production device 20 includes a partial oxidation device 40 and a finishing device 44 in fluid communication with the partial oxidation device 40. Various methods and processes for partial oxidation and finishing are known in the art, e.g., homogeneous partial oxidation, fully catalytic partial oxidation, autothermal reforming or fully catalytic steam methane reforming. It is envisioned that other synthesis gas production devices also may be used in accordance with the present invention. The partial oxidation device 40 also may include a start-up device, such as a pilot burner or igniter (not shown).

In a preferred embodiment, the partial oxidation device 40 is a gas-phase partial oxidation device in which the reactants, i.e., the hydrocarbon feed stream 8, the oxidation gas feed stream 56, and/or the aqueous stream 62, enter the partial oxidation device 40 in a premixed state. The finishing device 44 preferably is a catalytic finishing zone in which residual hydrocarbons are converted to synthesis gas. Conventional reforming catalysts may be used in the finishing device 40. Alternatively, a catalyst based on a structured support may be used.

The synthesis gas typically leaves the partial oxidation device 40 and enters a first portion of the finishing device 44 ′ at a temperature from about 900° C. to about 1400° C. The partial oxidation device 40 and the finishing device 44 are thermally linked by direct fluid connection, e.g., through a distributor plate, so that heat from the exothermic gas phase reaction in the partial oxidation device 40 is used to drive the endothermic steam reforming reactions in the finishing device 44. Furthermore, the second portion of the finishing device 44″ depicted in FIG. 2 also circumferentially surrounds the outer walls of the partial oxidation device 40, the reactant gas preparation device 32 and the first portion of the finishing device 44′. This design further enhances thermal integration and also shields the remainder of the apparatus 4 from the high temperatures experienced in the partial oxidation device 40.

The residence time of the synthesis gas in the partial oxidation device 40 and the finishing device 44 may be controlled to maximize conversion of the hydrocarbon feed stream 8 to obtain an optimum ratio of hydrogen to carbon monoxide, while also avoiding soot formation in the partial oxidation device 40 and coke formation in the finishing device 44. Preferably the residence times are from about 1 millisecond to about 1 second in the partial oxidation device 40 and from about 200 milliseconds to about 4 seconds in the finishing device 44.

In the embodiment shown in FIG. 2, the reactant gas preparation device 32, the partial oxidation device 40, and the first portion of the finishing device 44′ are centrally located about an axis “Y” and share a common wall 46. The remaining portion of the finishing device 44″ circumferentially surrounds wall 46 and is contained by wall 47. Walls 46 and 47 may be constructed from any suitable high temperature material such as high temperature ceramic or alloy. Alternatively or additionally, wall 46 and/or wall 47 may be actively cooled internally. Because the reactant preparation device 32, the partial oxidation device 40, and the finishing device 44 are contained within the pressure vessel 16, walls 46 and 47 do not have to be constructed of materials rated for the full system pressure. Preferably, walls 46 and 47 are fixed within the pressure vessel 16 only on the top end, like wall 47, or the bottom end, like wall 46, in order to allow for free axial thermal expansion of the walls.

As shown in FIG. 2, the finishing device 44 is in fluid communication with a synthesis gas conditioning device 24 that circumferentially surrounds wall 47 and is contained by wall 65. The synthesis gas conditioning device 24 cools the synthesis gas from the finishing device 44 to approximately the range of temperatures required for subsequent conversion in the synthesis gas conversion device 28. Typically, for a Fischer-Tropsch based conversion process, the synthesis gas enters the synthesis gas conditioning device 24 at a temperature from about 800° C. to about 1000° C., and subsequently is cooled in the synthesis gas conditioning device 24 to from about 180° C. to about 300° C. Preferably, the synthesis gas is cooled to a temperature of between about 190° C. to about 250° C.

The synthesis gas conditioning device 24 is thermally integrated with the reactant gas preparation device 32 in two ways: it includes a first synthesis gas heat exchanger 48 that preheats the oxidation gas feed stream 56 in fluid communication with the reactant gas preparation device 32 while cooling the synthesis gas stream; and it includes a second synthesis gas heat exchanger 60 that preheats an aqueous stream 62, which may be used to supply steam reactant to the reactant preparation device 32 and/or to preheat the hydrocarbon feed stream 8 while cooling the synthesis gas stream. Additionally, there may be a third heat exchanger (not shown) to preheat hydrocarbon feed stream 8 in fluid communication with the reaction preparation device 32. This thermal integration not only conserves energy but also eliminates the need to include one or more separate preheating devices for the oxidation gas feed stream 56 and/or the hydrocarbon feed stream 8, and serves to cool the synthesis gas stream.

The illustrated synthesis gas conditioning device 24 includes a first synthesis gas heat exchanger 48 which includes a first hollow body 52 that circumferentially winds downward about the finishing device 44. The oxidation gas feed stream 56 is introduced into the pressure vessel 16 through conduit 57, and travels through the first hollow body 52, terminating at the reactant gas preparation device 32 at inlet 56′. The first synthesis gas heat exchanger 48 exchanges heat through the first hollow body 52 into the oxidation gas feed stream 56. Alternatively, the hydrocarbon feed stream may be preheated in the first synthesis gas heat exchanger (not shown). Yet another alternative is to premix the hydrocarbon feed stream 8 and the oxidation gas feed stream 56, and then preheat the premixed stream in the first synthesis heat exchanger 48 (also not shown). Yet another alternative is to premix the hydrocarbon feed stream 8, the oxidation gas feed stream 56, and the aqueous stream 62, and then preheat the premixed stream in the first synthesis gas heat exchanger 48 (also not shown).

The synthesis gas conditioning device 24 also includes a second synthesis gas heat exchanger 60 which includes a second hollow body 64 that circumferentially winds around the reactant gas preparation device 32 about the bottom end of the synthesis gas conditioning device 24. The second synthesis gas heat exchanger 60 exchanges heat through the second hollow body 64 from the synthesis gas into aqueous stream 62. The aqueous stream 62 is preheated and preferably vaporized in this manner and may be used to provide steam reactant to the reactant gas preparation device 32 for pre-mixing and/or used to preheat the hydrocarbon gas feed stream 8 and/or the oxidation gas feed stream 56. The latter may be achieved as described above, i.e., by passing the preheated aqueous stream 62 through a coil, tube, or finned-tube heat exchanger (not shown) located in the reactant gas preparation device 32.

Preferably, as shown in FIG. 2, the second synthesis gas heat exchanger 60 is positioned so that synthesis gas exiting from the finishing device 44 first flows past the second synthesis gas heat exchanger 60. This configuration is preferred because vaporizing an aqueous stream typically will provide a greater rate of heat exchange than will sensible preheating of an oxidation and/or a hydrocarbon feed stream. Alternatively, the first synthesis gas heat exchanger may be intertwined with the second synthesis gas heat exchanger (not shown). The first hollow body 52 and the second hollow body 64 may be constructed of any suitable alloy. For example, stainless steel or a nickel-based alloy such as hastelloy, may be used. Alternatively or additionally, a direct quench may be used, wherein a liquid, e.g., water, is introduced directly into the synthesis gas that extracts heat from the synthesis gas when it is converted to steam.

As shown in FIG. 2, wall 65 is disposed circumferentially about the synthesis gas conditioning device 24. The wall 65 may be constructed of any suitable material, including carbon steel. The interior surface of wall 65 also may be lined with an insulation material, e.g., ceramic. Similar to walls 46 and 47, wall 65 need not be constructed for full system pressure rating as the wall 65 is within the pressure vessel 16. Similar to walls 46 and 47, wall 65 preferably is fixed within the pressure vessel 16 only at one end to allow for free axial thermal expansion of the wall.

In yet another alternative embodiment of the invention, the synthesis gas conditioning device 24 contains a device (not shown) to introduce water directly into the synthesis gas stream to cool the synthesis gas stream, thus reducing the need for other types of heat exchange.

Again referring to FIG. 2, circumferentially surrounding the synthesis gas conditioning device 24 is a synthesis gas conversion device 28 that includes a catalytic bed reactor 68 and one or more reactor heat exchangers 72. Alternatively, the reactor may take the form of a staged catalyst bed reactor and the heat exchanger may take the form of intermediate heat removal zones. In another alternative embodiment, the reactor may take the form of a graded catalyst bed with heat exchange taking the form of graded heat removal zone. In yet another alternative design, the synthesis gas conversion device includes a reactor where the catalyst is packed inside hollow tubes and the heat transfer medium flows around the tubes. For reactor technology such as three-phase slurry bubble columns, ebulliating bed, or fluidized bed operation, the flow pattern of the entire system may be configured so that the synthesis gas would enter the reactor from the bottom of the reactor and flow up through the synthesis gas conversion device 28.

Synthesis gas conversion may be accomplished by catalytic conversion of the conditioned synthesis gas to liquid products, e.g., by a Fischer-Tropsch reaction. Besides hydrocarbon liquid product, other desirable liquid products, include, but are not limited to, methanol, ethanol, dimethyl ether, ammonia, and alpha-olefins. Various reactor technologies known in the art may be used, e.g., fixed bed, slurry bed, or in the embodiment depicted in FIG. 2, a down-flow fixed-bed Fischer-Tropsch synthesis reactor. Catalysts suitable for use in accordance with the present invention are known and may be packed either as a conventional supported packed bed or a structured bed. For applications using Fischer-Tropsch catalyst technology, either cobalt-containing or iron-containing catalysts may be used. Since the selectivity of iron-based and cobalt-based Fischer-Tropsch catalysts are sensitive to reaction temperature, heat regulation, typically heat removal, is necessary because Fischer-Tropsch reactions are highly exothermic.

The reactor 68 generally includes an inlet reaction zone 84 and an outlet reaction zone 88. Typically, the rates of heat exchange in these two zones are different because both the rate of heat generation and the efficiency of heat conduction and removal vary significantly over the length of the bed. This is due in part to a variation in reactant available and product concentrations present throughout the reactor bed. Typically, the rate of heat exchange required in the inlet reaction zone 84 is significantly greater than that required in the outlet reaction zone 88 because of the higher concentration of gaseous reactants and lower concentration of liquid products in the inlet reaction zone 84 relative to the same concentration of the same present in the outlet reaction zone 88.

In order to compensate for differentials in heat exchange requirements in the reactor 68, the reactor heat exchanger(s) 72 may be configured differently in the inlet and outlet reaction zones 84, 88. As shown in FIG. 2, the reactor heat exchanger 72 includes an inlet hollow body 92 in the inlet reaction zone 84 and an outlet hollow body 96 in the outlet reaction zone 88, both passing through the reactor 68 in a serpentine fashion. The reactor heat exchanger 72 exchanges heat through the inlet hollow body 92 and the outlet hollow body 96 into an inlet fluid stream 86 and an outlet fluid stream 90, respectively. Preferably the fluid streams 86, 90 are aqueous, so steam may be generated within the reactor heat exchanger 72. The depicted inlet hollow body 92 and the outlet hollow body 96 are coils. Alternatively, the hollow bodies may be constructed of other conventional heat transfer elements such as tubes or plates. Optionally, the hollow bodies may have shapes that are not circular in cross-section and/or have extended surface features, e.g., fins. The inlet hollow body 92 and the outlet hollow body 96 may be constructed from any suitable material, such as stainless steel.

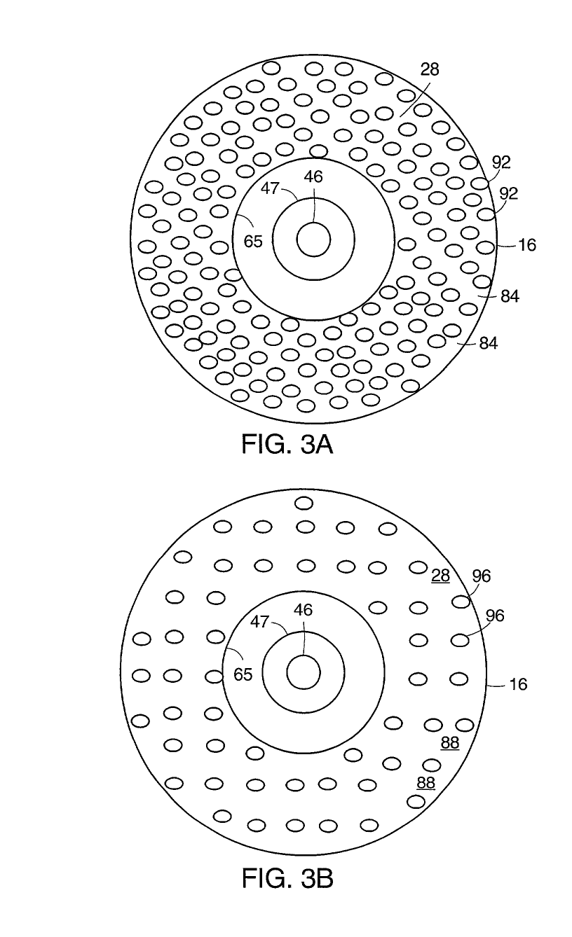

FIGS. 3A and 3B are cross-sectional schematic views of FIG. 2, taken along lines A-A and B-B, respectively. FIG. 3A depicts a cross-section view of the inlet reaction zone 84 and the inlet hollow body 92. FIG. 3B depicts a cross-sectional view of the outlet reaction zone 88 and the outlet body 96. The inlet hollow body 92 defines an effective outer surface area A_{Inlet} for heat exchange disposed in the inlet reaction zone 84, and the outlet hollow body 96 defines an effective outer surface area A_{Outlet} disposed in the outlet zone 88. A_{Inlet} and A_{Outlet} are chosen to match the heat extraction requirements for particular type of synthesis gas conversion device employed. As can be seen from a visual comparison of FIGS. 3A and 3B, the effective cross-sectional surface area of the inlet hollow body 92 is substantially greater than that of the outlet hollow body 96. That is, A_{Inlet} is greater than A_{Outlet}. This differential in effective outer surface area allows for a greater rate of heat exchange in the inlet reaction zone 84 than in the outlet reaction zone 88. Additionally, the effective outer surface area of the hollow bodies 92, 96 further may be increased by including fins or the like (not shown) to either or both of the inlet hollow body 92 and the outlet hollow body 96. Alternatively, heat extraction requirements may require more heat extraction in the outlet reaction zone 88 than that required in the inlet reaction zone 84. Accordingly, the inlet reaction zone 84 and the outlet reaction zone 88 may be configured such that A_{Inlet} is less than A_{Outlet}. Additionally or alternatively, the rate of heat exchange also may be controlled by independently increasing the pressures and/or the flow rates of the fluid streams 86, 90 in the inlet hollow body 92 and the outlet hollow body 96 so as to control the boiling point of the heat exchange fluid, which preferably is water.

Alternatively, a reactor may be divided into more than two reaction zones, each with its own heat exchange hollow body. For example, the reactor may have an inlet reaction zone, an outlet reaction zone and a third reaction zone disposed between the inlet reaction zone and the outlet reaction zone. The reactor heat exchanger in this case may include and inlet hollow body, an outlet hollow body and a third hollow body, respectively, each defining an effective outer surface area A_{Inlet}, A_{Outlet}, and A_{Third}, respectively designed to match the heat extraction requirements for the particular process used. The effective outer surface areas may be varied depending on the synthesis gas conversion device employed to better control the temperature within the synthesis gas conversion device. For example, the inlet hollow body, outlet hollow body, and third hollow body may be configured such that A_{Third} is greater than A_{Inlet}, and A_{Inlet} is greater than A_{Outlet}. Flowing through each would be an inlet fluid stream, an outlet fluid stream and a third fluid stream, respectively. The pressures of each fluid stream and the flow rate of each fluid stream also independently may be varied to further control the rate of heat exchange in each reaction zone.

FIG. 2 also depicts an optional product separation device 36 in fluid communication with the synthesis gas conversion device 28 for separating the liquid product stream 12 from a gaseous product stream 14. Product separation devices are known in the art and typically are based on gravimetric separation, taking advantage of density differences in the products, e.g., a flash drum or a settling tank. The illustrated product separation device 36 is configured similar to a settling tank with an orifice 76 located at the bottom to gravity drain the liquid products to a collection area 80. In FIG. 2, the product separation device 36 is incorporated as an integral part of the pressure vessel 16 and is disposed below the synthesis gas conversion device 28 to receive the liquid products 12 and to separate them from a gaseous product stream 14. In addition, for Fischer-Tropsch reaction applications, the cooling and separation of water-rich and hydrocarbon-rich liquid phases may be facilitated by increasing the residence time volume in the product separation device and including baffles housing cooling coils. Additionally, there may be separate liquid outlets for different liquid phases, e.g., the water-rich and the hydrocarbon-rich liquid phases.

The gaseous product stream 14 typically possesses some fuel value and may subsequently be used as fuel for any purpose, such as internal and/or external power consumption with a variety of power generation devices, e.g. a gas turbine. Additionally, constituents of the gaseous byproducts may be separated for other uses. For example, hydrogen may be separated from the gaseous product stream 14 and used in a sulfur removal device and/or a product upgrade device. Also, a portion of the tail gas or gaseous product stream 14 may be recycled to the synthesis gas production device 20 to increase the thermal efficiency of apparatus 4.

The apparatus 4 also may include a product upgrade device (not shown) in fluid communication or integrated with the synthesis gas conversion device 28 or with the product separation device 36. The product upgrade device may be used to improve the quality and/or purity of the liquid product 12. In the case of a Fischer-Tropsch-based synthesis gas conversion device, the upgrading device may be used to reduce the molecular weight of the heaviest product fraction, e.g., wax, by conventional processes such as hydrocracking, hydroisomerization or thermal cracking. The product upgrade device may be located either inside or outside the pressure vessel 16. Product upgrade devices typically include various conversion chemistries, e.g., catalytic hydroisomerization. Hydrogen reactant, necessary for hydroisomerization upgrading, may be separated from the gaseous product stream 14.

Optionally, inside the pressure vessel 16, at the exit of the synthesis gas conditioning device, or at the exit of the synthesis gas conversion device, a slip-stream of the synthesis gas containing a substantial amount of hydrogen may be diverted to separate hydrogen for use in other parts of the apparatus 4. For example, the hydrogen may be used as a feed stream for a hydrocarbon feed stream desulfurization device or a liquid product upgrade device. Such a separation may be effected through conventional separation technology, e.g., membrane technology or cryogenic separation. Preferably, membrane separation is used. The purified hydrogen then may be re-pressurized to system pressure, if necessary, and utilized. Subsequently, the residual synthesis gas can be returned to the synthesis gas stream.

The outer wall of the pressure vessel 16 typically is constructed of suitable grade steel, such as carbon steel, which is lined with a suitable insulating material, such as ceramic insulating material. The insulating material preferably is disposed inside the carbon steel. The pressure vessel 16 typically has a system pressure rating of from about 25 barg to about 100 barg. As discussed above, however, the interior vessel walls, such as walls 46, 47 and 65, need not be rated for the full system pressure, resulting in a significant cost savings. Furthermore, the outer wall of the pressure vessel 16 may be constructed of materials rated for the operating temperature of the synthesis gas conversion device 28, typically between about 180° C. and 400° C. for a Fischer-Tropsch based conversion process. The temperature rating of the steel may be lower than these temperature ranges if it is lined with an insulating material.

In the embodiment in FIG. 2, the partial oxidation device 40, the finishing device 44, the synthesis gas conditioning device 24, and the synthesis gas conversion device 28 are in a nested configuration. As shown, the partial oxidation device 40 and the finishing device 44 are centrally disposed within the pressure vessel 16. The synthesis gas conditioning device 24 surrounds the partial oxidation device 40 and the finishing device 44, and the synthesis gas conversion device 28 surrounds the synthesis gas conditioning device 24.

The finishing device 44 may be disposed in relation to the partial oxidation device 40 as shown in FIG. 2. That is, the reactant gas preparation device 32, the partial oxidation device 40 and the first portion of the finishing device 44′ may be disposed in series along a central axis Y, with the remaining portion of the finishing device 44″ circumferentially surrounding the reactant gas preparation device 32, the partial oxidation device 40 and the first portion of the finishing device 44′ as shown. Alternatively, the partial oxidation device 40 may extend substantially through the pressure vessel 16 along its central axis Y, and in this case, the finishing device 44 would only circumferentially surround the partial oxidation device 40 (not shown).

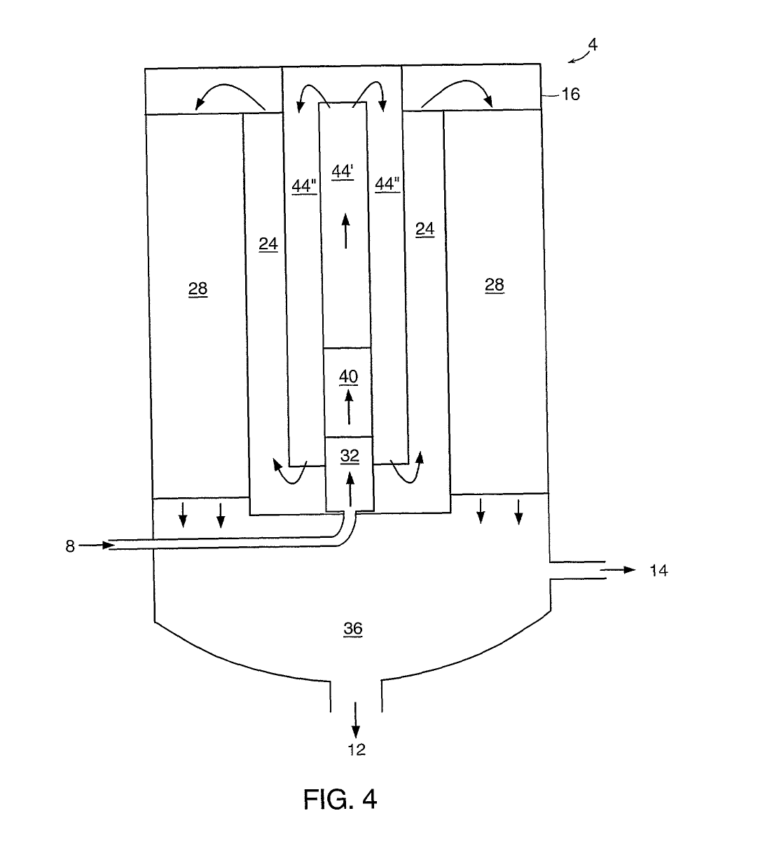

FIGS. 4, 5 and 6 are high-level cross-sectional schematic views of alternative embodiments of apparatus 4 for conversion of a hydrocarbon feed stream 8 into liquid products 12 in accordance with the present invention. FIGS. 4, 5 and 6 depict apparatus 4 that include a pressure vessel 16 that generally includes a reactant gas preparation device 32, a partial oxidation device 40, a finishing device 44, a synthesis gas conditioning device 24, and a synthesis gas conversion device 28.

FIG. 4 shows a high-level cross-sectional schematic view of the embodiment depicted in FIG. 2. As is described for FIG. 2, the hydrocarbon feed stream 8 enters through the lower end of the pressure vessel 16 and generally flows upward through the reactant gas preparation device 32 and through the partial oxidation device 40 where it is partially oxidized. The partially oxidized hydrocarbon feed stream flows upward through the first portion of the finishing device 44′ and then downward through the remaining portion of the finishing device 44″. The resulting synthesis gas flows upwardly through the synthesis gas conditioning device 24 and is subsequently converted to liquid products in the down-flow synthesis gas conversion device 28. Finally, the products of the conversion flow downward through the product separation device 36 and the liquid products 12 exit the apparatus 4 at the bottom of the pressure vessel 16, and the gaseous product stream 14 exit the pressure vessel near the top of the separation device 36.

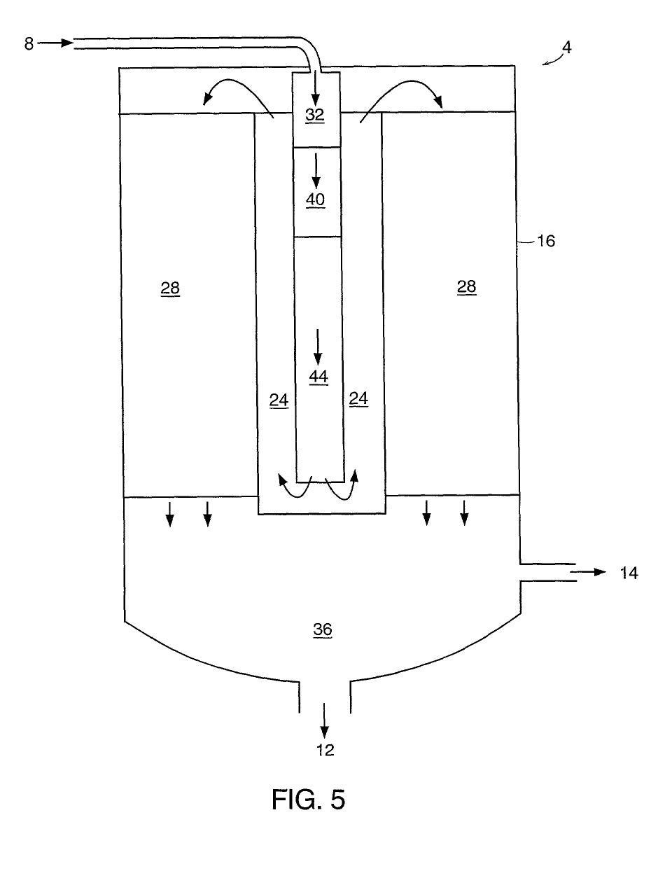

In FIG. 5, the hydrocarbon feed stream 8 enters through the top of the pressure vessel 16 and generally flows downward through the reactant gas preparation device 32, through the partial oxidation device 40 where it is partially oxidized, and then through the finishing device 44. The resulting synthesis gas flows upwardly through the synthesis gas conditioning device 24 and is subsequently converted in the down-flow synthesis gas conversion device 28. Finally, the conversion products flow downward through the product separation device 36 and the liquid products 12 exit the apparatus 4 at the bottom of the pressure vessel 16, and the gaseous product stream 14 exits the pressure vessel near the top of the separation device 36.

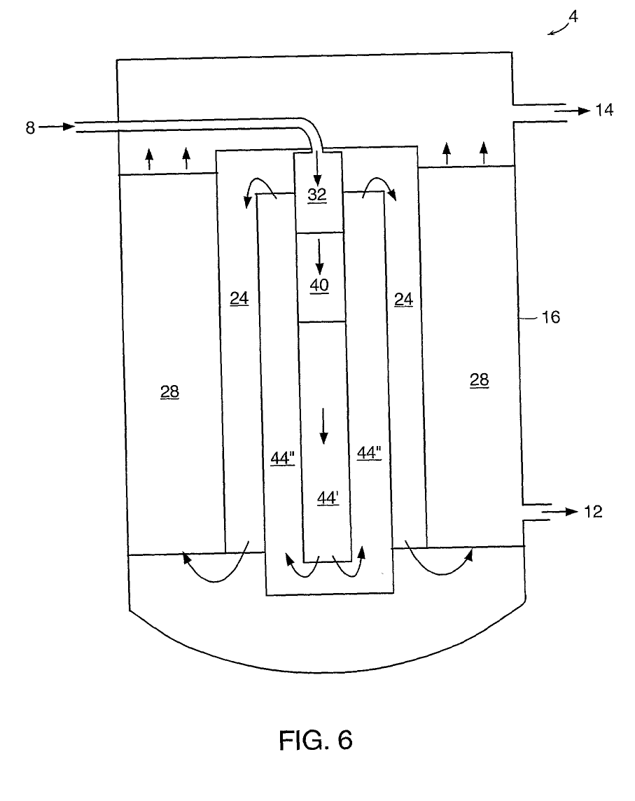

In FIG. 6, the hydrocarbon feed stream 8 enters through the top end of the pressure vessel 16 and generally flows downward through the reactant gas preparation device 32, through the partial oxidation device 40 where it is partially oxidized. Subsequently, the partially oxidized hydrocarbon feed stream flows downward through the first portion of the finishing device 44′ and upward through the remaining portion of the finishing device 44″. The resulting synthesis gas then flows downwardly through the synthesis gas conditioning device 24 and is subsequently converted to liquid products in an up-flow synthesis gas conversion device 28. Finally, the liquid products 12 exit the apparatus 4 at a conduit located about the bottom end of the synthesis gas conversion device 28 and gaseous product stream 14 exits the apparatus 4 at the top of the pressure vessel 16. For this embodiment, the synthesis gas conversion device may be an ebulliating, fluid bed, or a three-phase slurry bubble column reactor.

The axially symmetric integration of the processing devices shown in the Figures minimizes internal separations between each device and thereby minimizes the overall size of the apparatus. Furthermore, axially symmetric integration results in a more structurally sound and safer apparatus because the internal walls are subjected to lower pressure differentials compared to a design where each device is in its own pressure vessel. Accordingly, such a design is preferred, however non-axially symmetric designs are contemplated as well.

Control of the system temperatures and pressures largely follow conventional control practice. For example, the heat exchangers in the apparatus are designed with surface areas and heat transfer characteristics to allow thermal balance within the system based on conventional heat exchanger design principles. Similarly, the reactor vessels and conduits within the system are designed in such a way as to result in only a modest pressure drop throughout the system. Control of the system pressure may be achieved by regulating the inlet pressure of the hydrocarbon feed stream 8, the oxidation gas feed stream 56, and the aqueous stream 62, and the flow rate of the liquid product stream 12 and the gaseous product stream 14. The temperature in the partial oxidation zone of the system also may be controlled by adjusting the ratios of the hydrocarbon feed stream 8, the oxidation gas feed stream 56, and the aqueous stream 62. Temperatures in the synthesis gas conditioning device 24, the synthesis gas conversion device 28, the product separation device 36, and the product upgrade device further may be controlled by regulating the flow rates and pressures of the heat exchange fluid streams in these devices. Preferably, the apparatus 4 allows independent variation of the heat exchange fluid pressures and flow rates in each heat exchange device in order to achieve greater control of each process temperature as well as a faster response time. Start-up of the apparatus 4 follows conventional practice, which generally involves gradually heating up the apparatus 4 using the apparatus heat exchangers in addition to preparing the catalysts by reducing them with a low concentration of hydrogen in an inert gas stream, e.g., between about 1% and 10% hydrogen.

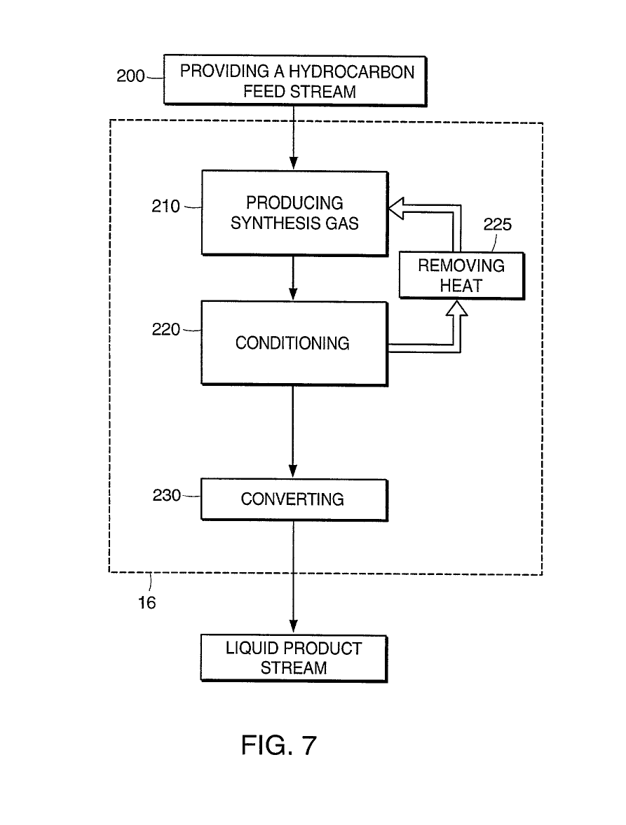

FIG. 7 is a flowchart summarizing an embodiment of a method of the present invention for conversion of a hydrocarbon feed stream into a liquid product stream. Broadly, a method of the invention includes the steps of: (a) providing a hydrocarbon feed stream (Step 200); (b) producing a synthesis gas stream from the hydrocarbon feed stream in a synthesis gas production device (Step 210); (c) conditioning the synthesis gas stream (Step 220); and (e) converting the synthesis gas stream to a liquid product stream (Step 230). The conditioning step includes: (c′) removing heat from the synthesis gas stream through a first hollow body into a reactant feed stream passing through the first hollow body to provide a preheated reactant feed stream, and (c″) feeding the preheated reactant feed stream into the synthesis gas production device (Step 225).

Although generally discussed directly above, practice of and methods encompassed by the invention have been further described in discussing the apparatus of the invention above. It should be understood that various configurations of devices of the apparatus permit the invention to be practiced in a number of ways. Accordingly, methods of the invention have been described above with reference to preferred embodiments, however, other methods are contemplated as within the scope of the invention. The following description is directed to further preferred embodiments of the methods of the invention.

Step 210 may be accomplished as discussed above. Preferably, synthesis gas is produced by partial oxidation in a partial oxidation device, followed by finishing in a finishing device. The method optionally may include the step of preheating the hydrocarbon feed stream and/or the oxidation gas feed stream prior to partially oxidizing the hydrocarbon feed stream. As discussed above, the hydrocarbon feed stream and/or the oxidation gas feed stream may be heated in a reactant preparation device located inside or outside the pressure vessel. Reactant preheating devices suitable for use in preheating the hydrocarbon feed stream are discussed above.

If the hydrocarbon feed stream comprises an impurity or contaminant, e.g., sulfur, the method may include the step of removing a substantial amount of the impurity from the hydrocarbon feed stream prior to partially oxidizing the hydrocarbon feed stream by incorporating the devices discussed above. The method also may include the step of pre-mixing the hydrocarbon feed stream, the oxidation gas feed stream and/or the aqueous stream to achieve the ratios discussed above.

Step 220 may be achieved using a synthesis gas conditioning device as described previously. Step 220 also may include the step of removing heat from the synthesis gas stream through a second hollow body into an aqueous stream as described above in reference to FIG. 2. The aqueous stream exiting the second hollow body may then be fed to a reactant preparation device for reactant preheating and/or mixing with the reactant feed stream prior to the conversion of the reactant gases to synthesis gas. As described above Step 220 also may include a direct quench as described above for FIG. 2.

Step 230 may be achieved by using the synthesis gas conversion devices described above. As discussed above, the conversion of the synthesis gas stream in Step 230 may occur in two zones of the reactor, i.e., in an inlet reaction zone and an outlet reaction zone. Step 230 may further include the step of removing the heat evolved in Step 230 using a reactor heat exchanger. As discussed above in greater detail, the reactor heat exchanger may include: an inlet hollow body defining an effective outer surface area A_{Inlet} disposed in the inlet reaction zone that exchanges heat through the inlet hollow body into an inlet fluid stream; and an outlet hollow body defining an effective outer surface area A_{Outlet} disposed in the outlet reaction zone that exchanges heat through the outlet hollow body into an outlet fluid stream. Preferably, A_{Inlet} is greater than A_{Outlet} to effect greater rates of heat exchange in the inlet reaction zone than in the outlet reaction zone.

Optionally, as discussed in greater detail above, the reactor may be divided into more than two reaction zones and contain more than two hollow bodies, each disposed in its respective reaction zone and defining its own surface area for greater control of heat exchange rates in the reactor. For example, the reactor may further comprise a third reaction zone disposed between the inlet reaction zone and the outlet reaction zone. In this embodiment, the reactor heat exchanger typically includes a third hollow body defining an effective outer surface area A_{Third} disposed in the third reaction zone that exchanges heat through the third hollow body into an third fluid stream. The surface areas of the inlet hollow body, the outlet hollow body and the third hollow body may be designed to meet different heat extraction requirements in each zone. For example, the hollow bodies may be designed such that A_{Inlet} is greater than A_{Third}, and A_{Third} is greater than A_{Outlet}.

Alternatively or additionally, the method may further include the steps of providing fluid streams to the hollow bodies at different pressures and flow rates in order to control the amount of heat extraction in each. Preferably, the pressure and flow rates in each hollow body may be independently varied. For example, a first fluid stream may be provided to the inlet hollow body at a first pressure P_{Inlet}, and a second fluid stream may be provided to the outlet hollow body at a second pressure P_{Outlet}. Additionally or alternatively, the fluid stream provided to the inlet hollow body may be provided at a greater flow rate than the fluid stream provided to the outlet hollow body to effect greater heat extraction in the inlet reaction zone.

Because gaseous products may be entrained in the liquid product stream, the method may include the step of separating a gaseous product stream from the liquid product stream. Such separation may be effected using a product separation device as discussed above in reference to FIG. 2. In addition, methods of the invention may include the step of upgrading the liquid product stream that may be effected using a product upgrade device as described above.

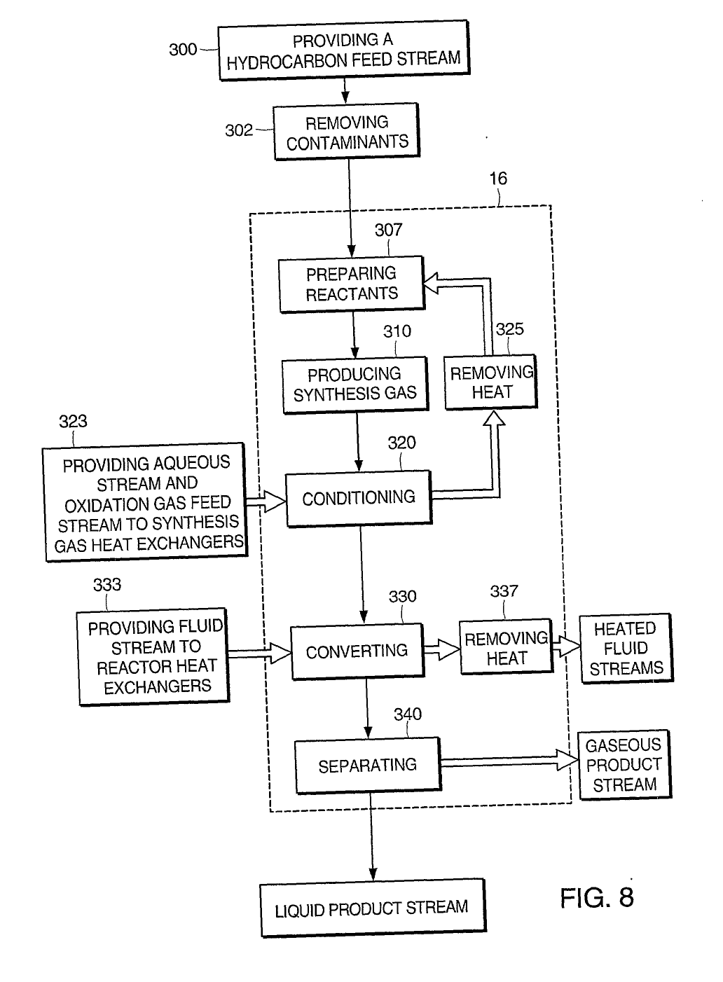

FIG. 8 is a flowchart summarizing another embodiment of a method for conversion of a hydrocarbon feed stream into a liquid product stream in accordance with the present invention. This embodiment includes the steps of: providing a hydrocarbon feed stream (Step 300); removing contaminants from the hydrocarbon feed stream (Step 302); preparing a reactant feed stream comprising the hydrocarbon feed stream, an aqueous stream, and an oxidation gas feed stream for synthesis gas production (Step 307); producing a synthesis gas stream from the reactant feed stream, (Step 310); conditioning the synthesis gas stream (Step 320); providing the aqueous stream and the oxidation gas feed stream to one or more synthesis gas heat exchangers (Step 323); removing heat from the synthesis gas stream by heat transfer to the aqueous stream and the oxidation gas feed stream (Step 325); converting the synthesis gas stream to a liquid product stream and a gaseous product stream (Step 330); providing one or more fluid stream to one or more reactor heat exchangers (Step 333); removing heat from the synthesis gas stream, liquid product stream, and gaseous product stream by heat transfer to the fluid stream (Step 337); and separating the liquid product stream from the gaseous product stream (Step 340). Further depicted in FIG. 8 that Steps 307, 310, 320, 325, 330 337, and 340 occur within a pressure vessel 16. The steps depicted in FIG. 8 have been described above. Preferably, Step 307 includes premixing and preheating the reactant gas feed stream as described above. The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. An apparatus for conversion of a hydrocarbon feed stream into a liquid product stream comprising:
a pressure vessel comprising:
a synthesis gas production device;
a synthesis gas conditioning device in fluid communication with the synthesis gas production device; and
a synthesis gas conversion device in fluid communication with the synthesis gas conditioning device,
wherein the synthesis gas production device and the synthesis gas conditioning device are nested within the synthesis gas conversion device.

2. The apparatus of claim 1 further comprising a reactant gas preparation device in fluid communication with the synthesis gas production device.

3. The apparatus of claim 2 wherein the reactant gas preparation device is within the pressure vessel.

4. The apparatus of claim 2 wherein the reactant gas preparation device comprises:
a sulfur removal device; and
a hydrocarbon feed stream preheating device in fluid communication with the sulfur removal device.

5. The apparatus of claim 1 further comprising a product separation device in fluid communication with the synthesis gas conversion device.

6. The apparatus of claim 1 further comprising a product upgrade device in fluid communication with the synthesis gas conversion device.

7. The apparatus of claim 1 wherein the synthesis gas conversion device comprises a product upgrade device.

8. The apparatus of claim 1 wherein the synthesis gas production device comprises:
a partial oxidation device; and
a finishing device in fluid communication with the partial oxidation device.

9. The apparatus of claim 1 wherein the synthesis gas conditioning device comprises:
a first synthesis gas heat exchanger disposed in the synthesis gas conditioning device that exchanges heat through a first hollow body into a reactant feed stream; and
a second synthesis gas heat exchanger disposed in the synthesis gas conditioning device that exchanges heat through a second hollow body into an aqueous stream.

10. The apparatus of claim 9 wherein the first synthesis gas heat exchanger is in fluid communication with the synthesis gas production device, such that the reactant feed stream is introduced into the synthesis gas production device.

11. The apparatus of claim 8 wherein the partial oxidation device, the finishing device, the synthesis gas conditioning device, and the synthesis gas conversion device are in a nested configuration such that the partial oxidation device and the finishing device are centrally disposed within the pressure vessel, the synthesis gas conditioning device surrounds the partial oxidation device and the finishing device, and the synthesis gas conversion device surrounds the synthesis gas conditioning device.

12. The apparatus of claim 11 wherein the finishing device surrounds the partial oxidation device.

13. The apparatus of claim 1 wherein the synthesis gas conversion device comprises:
a reactor comprising:
an inlet reaction zone, and
an outlet reaction zone; and
a reactor heat exchanger comprising:
an inlet hollow body defining an effective outer surface area A_{Inlet} disposed in the inlet reaction zone that exchanges heat through the inlet hollow body into an inlet fluid stream, and
an outlet hollow body defining an effective outer surface area A_{Outlet} disposed in the outlet reaction zone that exchanges heat through the outlet hollow body into an outlet fluid stream,
wherein A_{Inlet} is not equal to A_{Outlet}.

14. The apparatus of claim 13 wherein:
the reactor further comprises a third reaction zone disposed between the inlet reaction zone and the outlet reaction zone, and
the reactor heat exchanger further comprises a third hollow body defining an effective outer surface area A_{Third} disposed in the third reaction zone that exchanges heat through the third hollow body into an third fluid stream,
wherein A_{Inlet} is not equal to A_{Third}, A_{Third} is not equal to A_{Outlet}, and A_{Outlet} is not equal to A_{Inlet}.

15. An apparatus for conversion of a hydrocarbon feed stream into a liquid product stream, the apparatus comprising:
a pressure vessel comprising:
(a) a synthesis gas production device comprising:
a partial oxidation device, and
a finishing device in fluid communication with the partial oxidation device;
(b) a synthesis gas conditioning device in fluid communication with the synthesis gas production device, the synthesis gas conditioning device comprising:
a first synthesis gas heat exchanger disposed in the synthesis gas conditioning device that extracts heat through a first hollow body into an reactant feed stream, wherein the first synthesis gas heat exchanger is in fluid communication with the synthesis gas production device such that the reactant feed stream is introduced into the synthesis gas production device; and
a second synthesis gas heat exchanger disposed in the synthesis gas conditioning device that exchanges heat through a second hollow body into an aqueous stream;
(c) a synthesis gas conversion device in fluid communication with the synthesis gas conditioning device; and
(d) a product separation device in fluid communication with the synthesis gas conversion device,
wherein the partial oxidation device, the finishing device, the synthesis gas conditioning device, and the synthesis gas conversion device are in a nested configuration such that the finishing device surrounds the partial oxidation device, the synthesis gas conditioning device surrounds the finishing device, and the synthesis gas conversion device surrounds the synthesis gas conditioning device.

16. The apparatus of claim 15 wherein the pressure vessel further comprises:
a reactant gas preparation device in fluid communication with the synthesis gas production device comprising:
a reactant feed stream preheating device; and
a sulfur removal device in fluid communication with the reactant feed stream preheating device.

17. A method for conversion of a hydrocarbon feed stream into a liquid product stream comprising the steps of:
(a) providing a hydrocarbon feed stream;
(b) producing a synthesis gas stream from the hydrocarbon feed stream in a synthesis gas production device;
(c) conditioning the synthesis gas stream, wherein said conditioning step comprises:
(c′) removing heat from the synthesis gas stream through a first hollow body into a reactant feed stream passing through the first hollow body to provide a preheated reactant feed stream;
(c″) feeding the preheated reactant feed stream into the synthesis gas production device; and
(d) converting the synthesis gas stream to a liquid product stream.

18. The method of claim 17 wherein the reactant feed stream comprises the hydrocarbon feed stream.

19. The method of claim 17 wherein the hydrocarbon feed stream comprises an impurity and the method further comprises the step of removing a substantial amount of the impurity from the hydrocarbon feed stream prior to step (b).

20. The method of claim 17 wherein step (c) further comprises the step of:
(c′″) removing heat from the synthesis gas stream through a second synthesis gas heat exchanger disposed in the synthesis gas conditioning device that exchanges heat through a second hollow body into an aqueous stream flowing through the second hollow body.

21. The method of claim 17 wherein step (d) occurs in a reactor comprising an inlet reaction zone and an outlet reaction zone, and the method further comprises the step of:
(e) removing heat evolved in step (d) using a reactor heat exchanger comprising:
an inlet hollow body defining an effective outer surface area A_{Inlet} disposed in the inlet reaction zone that exchanges heat through the inlet hollow body into an inlet fluid stream, and
an outlet hollow body defining an effective outer surface area A_{Outlet} disposed in the outlet reaction zone that exchanges heat through the outlet hollow body into an outlet fluid stream,
wherein A_{Inlet} is not equal to A_{Outlet}.

22. The method of claim 21 wherein
the reactor further comprises a third reaction zone disposed between the inlet reaction zone and the outlet reaction zone, and
the reactor heat exchanger further comprises a third hollow body defining an effective outer surface area A_{Third} disposed in the third reaction zone that exchanges heat through the third hollow body into an third fluid stream,
wherein A_{Inlet} is not equal to A_{Third}, A_{Third} is not equal to A_{Outlet}, and A_{Outlet} is not equal to A_{Inlet}.

23. The method of claim 17 wherein a gaseous by-product stream is entrained in the liquid product stream, and the method further comprises the step of:
separating the gaseous by-product stream from the liquid product stream.

24. The method of claim 17 further comprising the step of:
upgrading the liquid product stream.

25. An apparatus for conversion of a hydrocarbon feed stream into a liquid product stream comprising:
a pressure vessel comprising:
a means for producing synthesis gas;
a means for conditioning synthesis gas in fluid communication with the means for producing synthesis gas; and
a means for converting synthesis gas in fluid communication with the means for conditioning synthesis gas,
wherein the means for producing synthesis gas and the means for conditioning synthesis gas are nested within the means for converting synthesis gas.
